(19) United States
(12) Patent Application Publication
(10) Pub. No.: US 2002/0146446 A1
(43) Pub. Date: Oct. 10, 2002

(54) Surgical-medical dressing for the treatment of body burns and for wound healing which employs human umbilical vein endothelial cell conditioned medium for human cell growth used in the manufacture of the dressing

(76) Inventor: Denis Eon Solomon, Miami, FL (US)

Correspondence Address: DENIS EON SOLOMON, 233 SW 31ST ROAD, MIAMI, FL, 33129, US

(21) Appl. No.: 10/076,266

(22) Filed: Feb. 15, 2002

(30) Foreign Application Priority Data
Apr. 4, 2000 (GB) GB 0008079.6

(51) Int. Cl. A61K 9/70

(52) U.S. Cl. 424/446

(57) ABSTRACT

A surgical-medical dressing is described which uniquely uses a sandwich of two extracelluar matrices grown on a composite composed of gelatin-fibronectin-heparan sulfate.

The culture medium used to grow the two cell types (dermal fibroblasts and dermal microvascular endothelial cells forming the second extracellular matrix) is the conditioned medium (CM) obtained from human umbilical endothelial cells used to form the first extracellular matrix. All cells in tissue culture are detached using 5 mM EDTA leaving their secreted acellular matrix behind and intact.

This CM can also neutralize the enzyme DISPASE commercially used to detach cultured epithelial sheets (‘Cultured epithelial autografts’ (CEAs)) from the matrix on which the human epidermal cells, forming the sheets are grown. CEAs are clinically used in wound and burn management.

### BACKGROUND TO THE INVENTION

R. J. Goss writing in Clinical Orthopedics & Related Research (1980) suggested that the relative inadequacy of regeneration in warm-blooded vertebrates may be attributed to the precocity with which they tend to form dermal scars in healing wounds; scars that are believed to preclude blastema production.

Blastemas, usually defined as accumulations of dedifferentiated mesenchymal cells beneath wound epithelium, are responsible, for example, for the re-growth of fingertips in young children after accidental amputations distal to the last joint. Such spontaneous re-growths, Goss thought may be examples of true epimorphic regeneration and include new bone, fingernail and skin with fingerprints. Fingertip regeneration fails to occur however, if blastema formation is prevented, by the traditional treatment of suturing skin flaps over the fresh amputation stump.

Goss concluded, “From what has been learned of the mechanisms by which vertebrate regeneration is achieved, the experimental initiation of renewed growth on otherwise non-generating structures might logically involve the manipulation of wound healing so as to deflect the natural tendency for scar formation in the direction of blastema production”.

In wound healing, the explanation for the final collagen deposition in the form of scar tissue is related to tissue bleeding. Fibrin of the blood clot serves as a substrate for the attachment and in-growth of cells, mainly fibroblasts. By this mechanism, the original unstable fibrin clot or mechanically weak fibrin adhesions, are turned (i.e. collagenized) into permanent fibrotic structures by the activity of invading fibroblasts. Interference with the above procedure might lead to the formation of lesser amounts of scar tissue or none at all.

Present day bioengineered skin substitutes (for a review, see Jeffrey R. Morgan & Martin Yarmush in ‘Science and Medicine’ July/August (1997) issue, pages 6-15) have their limitations and normally need an autograft or allograft eventually. Wound coverage for patients with extensive bums has traditionally been provided by temporary grafts of cadaver skin. Since the development of techniques for tissue culturing human epidermal keratinocytes, several dermal analogs to support keratinocyte growth have been the subject of research studies. Earlier studies had revealed that grafts composed of cultured cells and biopolymers dissolved rapidly when transplanted to athymic mice and humans. Also split thickness grafts, the gold standard for wound closure was limited by their unavailability in patients with total body burns.

In addition to body bums, diabetic ulcers, venous ulcers, pressure sores are but a few examples which affect large populations of patients. Available methods of treatment have proven inadequate, hence bioengineered skin substitutes provide the present focus for clinical investigation and evaluation. ORGANOGENESIS INC., of Canton, Mass. employs a skin equivalent called Apligraf, with an epidermal layer of cultured keratinocytes and a dermal analog of cultured neonatal fibroblasts in a collagen gel.

ADVANCED TISSUE SCIENCES of La Jolla, Calif., are clinically testing Dermagraft which is a composite of allogenic neonatal fibroblasts grown in vitro on a bioabsorbable mesh. The above are but two examples.

Problems arise with this traditional treatment because the graft may not ‘take’ and grafts rarely attain the sensory qualities of normal skin.

Besides patients with body burns, characterized in ascending severity, as first, second, or third degree, wound closure needs to be taken into consideration. Surgical stitching with sutures leaves the wound to heal itself with the passage of time. This results in granulation tissue and consequential scarring. No precursor of the complex cellular healing process is ever added.

Questions need to be asked.

The first one is ‘Why immediately cover a wound bed with an autograft or allograft with its huge population of resident keratinocytes, when the epidermal-dermal junction has not yet regenerated; when the extracellular matrix (ECM), if at all present, is in no position to deal with such a large population of transplanted keratinocytes?’

It must be borne in mind that keratinocyte adhesion and migration is significantly regulated by ECM components, such as collagens, fibronectin and laminin. Since basement membrane formation is pivotal to epidermal stability, doesn't the ECM need to be regenerated first, before it can exert its regulation of the keratinocytes?

The second question is ‘What about collagen?’ (Type III in papillary dermis, Type I in the reticular dermis).

In the papillary dermis, Type III collagen and fine elastin fibers is a woven multi-directional matrix with a random orientation. In contrast, in the reticular dermis, collagen is perpendicular to the lines of stress, and elastin is more prominent.

Cells adhere to collagen matrices through ECM receptors-the integrins. Specific integrin heterodimers bind to collagen. Binding of integrins to the ECM stimulates intracellular signalling through integrin clustering and the formation of focal adhesion sites. Thus, collagen acts as a receptor ligand to stimulate cellular responses. Because these integrins are the direct contact between cells and the ECM, they might be important cellular mechanotransducers, and are thought to determine the organization of epidermal tissue into proliferating and differentiating compartments (Rennekampff et al (1996)). It is widely recognized that the thinner the allograft/autograft, the better the ‘take’. In other words, do not overload the wound bed.

The approach to be adopted in this invention consists of supplying the relatively non-immunogenic acellular ‘building blocks’ of the dermis to the wound bed or burn area, to act as a template for the attraction, promotion of migration, and colonization by the host's cells, e.g. dermal fibroblasts and dermal microvascular endothelial cells (HDMECs), but more importantly, to allow the cellular formulation of the subsequent architecture of the healing tissue. There will be no overloading of the wound bed, and the proposed dressing will be left undisturbed within/on the wound/ burn area.

In 1992, 1 authored a paper that linked the acellular sub-endothelial matrix, or ECM of human umbilical vein endothelial cells (HUVECs) with that of human aortic endothelial cells. One of the intriguing aspects of that study was the adherence of 11^{th} passage HUVECs (isolated from one umbilical cord) on to the prepared

ECM of 6^{th} passage HUVECs, isolated from another cord. This was illustrated by a cellular photomicrograph as FIG. 2. Most of the seeded cells had adhered within two hours, reached confluence in eighteen hours, and were positively characterized as endothelial cells.

Since then, I found a publication that suggests that a further linkage with the ECM of microvascular endothelial cells isolated from the dermis of neonatal and adult skin (Kramer et al (1985)). Furthermore, another paper (Sontheimer (1989)), suggests that newborn human dermal microvascular endothelial cells (HDMECs) share with human umbilical vein endothelial cells, (HUVECs), the following properties:

Capacity to bind T-cells after IL-1 stimulation¹.

To express Class II antigens after gamma-IF stimulation .

To present Class II antigens to unprimed allogenic CD4⁺ T-cells³. ¹Fleck R. M., Geppert T. D. & Sontheimer R. D. (1986) Gamma-interferon induction of class II surface antigens on cultured human dermal microvascular endothelial cells. Clin. Res. 34, 749 Abs. ²Haskard D. O., Cavender D., Fleck R. M., Sontheimer R. D. & Ziff M. (1987) Human dermal microvascular endothelial cells behave like umbilical vein endothelial cells in T-cell adhesion studies. J. Invest. Dermatol. 88, 340-344. ³Sontheimer R. D. (1989) Perivascular dendritic macrophages as immunobiological constituents of the dermal microvascular unit. J. Invest. Dermatol. 93(2), Suppl., 96S-101S.

Consider the table below which compares and contrasts the extracellular secretions from different cell types:

TABLE I

| Human | |
|---|---|
| endothelial cells | Extracellular Secretions |
| Aortic, adult | Type IV procollagen, |
| vena cava | thrombospondin and fibronectin |
| Umbilical vein | Type IV procollagen, |
| | thrombospondin and fibronectin |
| Neonatal/Adult | Type IV procollagen, |
| dermis | thrombospondin, fibronectin and |
| | laminin |
| **Neonatal/adult | **Laminin, collagen type IV, |
| skin fibroblasts | perlecan, and nidogen/entactin |
| *Epidermal | *Type IV procollagen, type VII |
| keratinocytes | collagen, fibronectin, plus laminin (at |
| | confluence) |

The only difference between the human neonatal/adult skin and the other endothelial cells is the additional secretion of laminin.

That these inter-relationships occur might have been expected, if one postulates a step further on, and considers the likelihood of another tissue inter-relationship, this time, with human bone marrow.

G. D. Winter, who suggested the concept of an occlusive dressing, studied allogenic skin grafts in pigs, and Kangesu et al (1993) opined that the dermal grafts acted as temporary templates, that attracted host mesenchymal cells, possibly analogous to the process of osteoinduction in bone grafts. Keating et al (1982) noted that following human bone transplantation, donor-derived spindle cells express factor Vil (an endothelial cell marker), in up to 25% of the transplanted cells, synthesize type IV collagen, and demonstrate the typical tight junction complexes of endothelial cells.

Karasek (1993) reported that under the influence of cytokines, generated by either chemical or physical injury, the skin microvascular endothelial cell retained a capacity to generate cells normally considered to be derived from bone marrow. As a result, he suggested that the vasculature might play a far more central role in the remodeling of injured tissue and in the etiology of fibrosis, than had been previously considered.

If that conclusion is true, then further proof is provided by the Langerhans cells of the human epidermis, which originate from bone marrow, and play an important role in the immune function of the skin. It is notable that only fetal skin and fetal bony tissue heal without scar formation; fetal wounds in muscles, tendons, and the gastrointestinal tract heal by fibrosis and contraction, as in the adult (Balazs & Larsen (2000)). Table 2 below illustrates the differences in wound repair between adult skin and fetal skin.

TABLE 2

| Adult skin | Fetal skin |
|---|---|
| Fibrin clot | No fibrin clot |
| Delayed deposition of fibronectin | Immediate fibronectin deposition |
| Acute inflammation-high levels of | Lack of inflammatory response-low |
| Growth factors | levels of growth factors |
| Slower response to injury site-‘activation’ of | Faster response-fetal fibroblasts need |
| fibroblasts-excessive fibroblast infiltration | no activation-different fibroblast |
| | phenotype |
| Macrophages recruited to wound site | Macrophages absent from wound site |
| Lower ratio of Type III/Type I collagen | Higher proportion of Type III/Type I |
| | collagen. Type III collagen has increased |
| | solubility, some in procollagen form |
| Massive collagen deposition in dense parallel | Less synthesis, less deposition in |
| bundles. | reticular fashion |
| Hyaluronic acid (HA) levels peak at 2-4 days, | Abundant HA levels; high for weeks-no |
| then fall rapidly. | lymphocyte adhesion |
| Collagenous scar-no normal skin-non-sterile | No scar formation-regenerative process- |
| conditions | sterile conditions |

*Compiled from:
Balazs E. A. & Larsen N. E. (2000)-Hyaluronan: Aiming for perfect skin regeneration in Scarless Wound Healing, Chapter 7, (Eds.) Hari G. Garg & M. T. Longaker, Marcel Dekker Inc., New York.
McCallion R. L. & Ferguson M. W. J. (1996)-Fetal wound healing and the development of antiscarring therapies for adult wound healing in The Molecular and Cellular Biology of Wound Repair, 2^{nd} edition, Chapter 18, (Ed.) Richard A. F. Clark, Plenum Press, New York.

Interestingly, during embryonic development, blood cells and endothelial cells differentiate from the same group of mesenchymal stem cells. The embryonic inter-relationships between blood cells and the endothelium as described by Wagner (1980) is shown below.

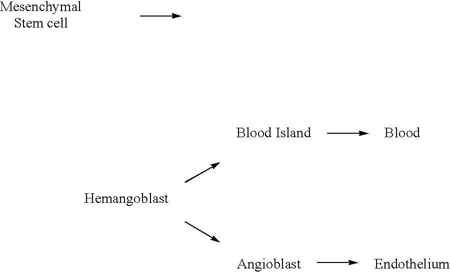

Fibroblasts are the principal collagen-producing cells in the heart producing the major fibrillar collagens, Types I and III (same as skin). In the papillary dermis of the skin, dermal fibroblasts secrete the ECM proteins and they are nourished by the skin capillaries/blood microvessels from which the dermal endothelial cells are extracted for tissue culture.

Thrombospondin (TS) is a platelet protein with lectin activity that is released from its storage sites in the alpha-granules by thrombin, binds to the fibrinogen associated with the activated platelet surface, and promotes platelet aggregation. It exhibits a selective affinity to Type V collagen in the ECM and TS may interact with fibrinogen, fibronectin, laminin, and heparin. Matrix-bound TS is secreted by endothelial cells but degraded, not by the action of a secreted protease, but intra-cellularly by fibroblasts. Fetal calf serum contains 3040 ug/ml. of TS, hence the usual conditions of tissue culture mimic a wound area, in the sense that TS is present in the serum-containing growth medium.

Laminin is a 500-kDa glycoprotein composed of three chains. It is a major component of basement membranes. Laminin stimulates cell adhesion, growth, differentiation, and is responsible for tubule formation in dermal microvascular endothelial cells. Laminin 5, a laminin isoform is secreted in copious amounts by keratinocytes and is one of the first ECM ligands deposited by migrating keratinocytes during in vivo wound healing, (Zhang & Kramer (1996)).

Fibronectin, is a 540-kDa glycoprotein dimer of two similar polypeptide chains with some variation induced by alternative splicing. It binds to Type III collagen, promotes cellular migration and may enhance basement membrane assembly. In fetal skin, the content of fibronectin is very high, as is the ratio of Type III/Type I collagen. As aging occurs this ratio decreases, as does the fibronectin content. Interestingly, in the fibrotic state, Type III/Type I collagen ratio is high also.

Hansbrough et al (1993) examined the growth of fibroblasts on polyglactin-910 (Vicryl), called Dermagraft. As the fibroblasts grew in the Vicryl mesh, they secreted proteins and glycoproteins resulting in the formation of an ECM that filled the matrix interstices. Levels of fibronectin were very high, resembling the levels in fetal skin. The ECM also contained collagens 1, III, VI, elastin, and decorin. The ratio of Type III/Type I collagen was not measured, thus no conclusion about the fibroblast phenotypic state can be drawn.

It is well known that gelatin binds to the —NH₂ terminus of fibronectin, and glycosaminoglycans e.g. heparan sulfate or dermatan sulfate would bind to the —COOH terminus. In both the papillary dermis and the reticular dermis, the ground substance is fibronectin and the lycosaminoglycans, hyaluronic acid, chondroitin4-sulfate, and dermatan sulfate. Glycosaminoglycans, (GAGs), are represented by disaccharide units of acidic (D-glucuronic/L-iduronic acid) and basic (D-galactosamine/D-glucosamine) sugar residues. Chondroitin sulfate B is also known as dermatan sulfate. The sulfation of the disaccharide units may vary and the linear GAGs themselves are covalently linked to a central ‘core’ protein to constitute a proteoglycan. Hyaluronic acid, (HA), is non-sulfated and is not attached to a ‘core’ protein.

Gelatin is thermally denatured collagen. At body temperature, gelatin is unpolymerized in a liquid state. Gelatin at neutral pH, at 4° C., polymerizes and forms a gel.

Hence, the composite gelatin-fibronectin-heparan sulfate will be prepared for use as the er er surface of the proposed surgical-medical dressing given in the ‘Laboratory Techniques’ section below.

### Proposed Acquisitions for the Invention

Human umbilical cords will be obtained from healthy patients (after proper donor screening for negative HIV, syphilis and hepatitis B) and stored in sterile tissue culture medium with antibiotics, and an anti-fungal agent. Human skin will be similarly stored. Tissue-culture medium for the growth of HUVECS will be purchased. Required growth factors for cell culture will be either purchased or prepared in the laboratory. Human fibronectin, gelatin, and the heparan sulfate will be purchased from recognized commercial suppliers.

### LABORATORY TECHNIQUES TO BE USED FOR THE PRODUCTION OF THE INVENTION PRODUCT

A composite will be prepared. This will consist of human fibronectin-gelatin-heparan sulfate. This composite will be the inner/lower surface of the proposed surgical-medical dressing. Human umbilical vein endothelial cells (HUVECs) will be isolated from a single umbilical cord, and cultured according to Jaffe et al (1973), using the above composite as a substrate. On reaching post-confluency, (approximately 9 days after seeding), these cells will be detached, using 5 mM EDTA leaving the intact sub-endothelial matrix (ECM) behind (Solomon 1992, 2002).

An aliquot of detached cells will be set aside and used for endothelial cell identity tests. The majority of the detached HUVECs will be reseeded on another composite and the process repeated. Hence, from one HUVECs population, (usually good for 12-14 non-enzymatic passages with a rubber policeman), several ECMs may be prepared. On the other hand, repeated growth on the same ECM and induced detachment will thicken the ECM.

The viability of the HUVECs ECM was found to be 4 weeks, if stored in an incubator at 37° C. in a 5% CO₂/95% air mixture. There is no doubt that the ECM may be pre-prepared and stored, for later use, as described below.

Human dermis will be mechanically abraded as the source of dermal microvascular endothelial cells (HDMECs) and dermal fibroblasts, as described by . These cells will be tissue-cultured on the HUVECs ECM, (adherence of both cell types is three hours after seeding) and monitored closely, since the fibroblasts can outgrow the endothelial cells. After a suitable period, depending on seeding population, these cells will be similarly detached, leaving a ‘mixed matrix’ behind, superposed on the HUVECs ECM. As above, repeated growth and induced detachment will thicken the mixed matrix left behind.

This surface will provide the er er surface of the proposed dressing, and epidermal keratinocytes will adhere to this surface (see below).

It can be foreseen that the proposed dressing is enclosed in a soft, pliable, gel-like substance which will dissolve over time in contact with the patient's skin. An antibiotic cornstarch spray may be used to compromise the normal blood coagulation pathway, before the application of the dressing, which will not be removed from the wound bed or burned skin surface.

Alternatively, if autologous skin is available, Normand & Karasek's (1995) publication, describing the isolation and serial propagation of keratinocytes, ECs, and fibroblasts from a single punch biopsy of human skin, will be employed with HUVECs CM as the culture medium for HDMECs

### SUMMARY

From my submitted 2002 manuscript, the facts given below are described.

The extracellular matrix of human umbilical endothelial cells (HUVECs ECM) was seeded with adult epidermal keratinocytes, human dermal microvascular endothelial cells (HDMECs), and dermal fibroblasts. Since all three types of cells and HUVECs share similar secreted glycoproteins, HUVECs could be routinely cultured using a soluble growth supplement specific for epidermal keratinocytes. Trypsinized keratinocytes rapidly adhered to HUVECs ECM, revealing an epithelial-mesenchymal interaction previously thought to be tissue-specific. Confluent epidermal keratinocytes triggered the release of a ‘ground substance’ perhaps providing an insight into ECM remodeling. The conditioned medium from HUVECs (HUVECs CM) was found to neutralize lingering after effects of Dispase. Hence, colonies of epidermal keratinocytes from stripped epidermis (from both fresh and cadaver skin tissue) in HUVECs CM adhered to HUVECs ECM. These colonies also comfortably proliferated on the mixed matrix secreted by HDMECs/dermal fibroblasts, as well as in a mixed culture of all three cell types on HUVECs ECM, gelatin or plastic. HUVECs CM was also used as the culture medium in the isolation and proliferation of HDMECs and dermal fibroblasts.

This proposed dressing DOES NOT CONTAIN any type of living cell and will be relatively non-immunogenic, since it only employs acelluar matrices, resting on a composite of natural materials.

### References

Goss R. J. (1980) Prospects for regeneration in man.

Clin. Orthop. & Rel. Res. 151, 270-282.

Jaffe E. A., Nachman R. L., Becker C. G. & Minick C. (1973) Culture of human endothelial cells derived from umbilical veins. Identification by morphologic and immunologic criteria. J. Clin. Invest. 52, 2745-2756. Rennekampff H. O., Kiessig V., & Hansbrough J. F. (1996) Research Review-Current concepts in the development of cultured skin replacements. J. Surg. Res. 62, 288-295.

Solomon D. E. (1992) The seeding of human aortic endothelial cells on the extra-cellular matrix of human umbilical vein endothelial cells. Int. J. Exp. Path 73, 491-501.

Cai J -P, Hudson S., Ye M. -W., & Chin Y. -H. (1996) The intracellular signaling pathways involved in MCP-1-stimulated T cell migration across microvascular endothelium. Cell. Immunol. 167, 269-275.

Wagner R. C. (1980) Endothelial cell embryology and growth. Adv. Microcirc. 9, 45.

Kramer R. H., Fuh G. M., Bensch K. G. & Karasek M. A. (1985) Synthesis of extracellular matrix glycoproteins by cultured microvascular endothelial cells isolated from the dermis of neonatal and adult skin. J. Cell. Physiol. 123, 1-9.

Winter G. D. (1975) Temporary skin cover in Basic problems in Burns, p.19-24, (Eds.) Vrabec R., Konickova Z. & Moserova J., Springer-Veriag, Berlin.

Karasek M. A. (1993) Phenotypic diversity and biologic potential of microvascular endothelial cells in vivo and in vitro, in Dermal Immune System, Chap. 8, p.149-162, (Ed.) Nickoloff Brian J., CRC Press, Boca Raton, Fla.

Sontheimer R. D. (1989) Perivascular dendritic macrophages as immunobiological constituents of the dermal microvascular unit. J. Invest. Dermatol. 93, no.2, Suppl., 96S-101S.

Zhang K. & Kramer R. H. (1996) Laminin 5 deposition promotes keratinocyte motility. Exp. Cell Res. 227, 309-322.

Hansbrough J. F., Morgan J. L., Greenleaf G. E. & Bartel R. (1993) Composite grafts of human keratinocytes grown on a polyglactin mesh-cultured fibroblast dermal substitute function as a brayer skin replacement in full-thickness wounds on athymic mice. J. Burn Care & Rehab. 14, no.5, 485494.

Kangesu T., Navsaria H. A., Manek S., Fryer P. R., Leigh I. M. & Green C. J. (1993) Kerato-dermal grafts: the importance of dermis for the in vivo growth of cultured keratinocytes. Br. J. Plastic Surg. 46, 401-409.

Keating A., Singer J. W., Killen P. D., Striker G., Salo A. C., Sanders J., Thomas E. D., Thoming D. & Fialkow P. J. (1982) Evidence for donor origin of the in vitro hematopoietic microenvironment following marrow transplantation in man. Nature 298, 280.

Benezra M., Vlodavsky I., Ishai-Michaeli R., Neufeld G., & Bar-Shavit R. (1993) Thrombin-induced release of active basic fibroblast growth factor-heparan sulfate complexes from subendothelial extracellular matrix. Blood 81, no.12, 3324-3331.

Normand J. & Karasek M. A. (1995) A method for the isolation and serial propagation of keratinocytes, endothelial cells, and fibroblasts from a single punch biopsy of human skin. In Vitro Cell. Dev. Biol.-Animal 31, 447455.

It is hoped that speedy wound closure would result from this new surgical-wound dressing. Even if this dressing were to later dissolve on application to a patient's wound, it would position, directly, non-cellular components of the human dermis exactly where they would be needed to aid wound healing. These non-cellular components of the human dermis consist primarily of extracellular proteins and collagen which have been shown to be relatively non-immunogenic. It is a known fact that that when physicians use a split-thickness graft, the so-called STSG, the degree of scarring and contracture of the grafted wound correlates inversely with the amount of dermis that is delivered in a STSG.

By using a mixed extracellular matrix, incorporated in this new dressing, composed of both endothelial cell and fibroblast non-cellular proteins, the healing growth of both epidermal and dermal cells will be actively encouraged.

It is envisaged that in the future, prior to any surgical procedure, which will require wound healing, a skin biopsy will be obtained. This will allow a standard gelatin/heparin substrate, overlaid with the extracellular matrix of human umbilical vein, to become seeded with the patient's own dermal endothelial cells and fibroblasts, which will provide an autologous mixed matrix extracelluar matrix.

Initially, it is proposed that sheets of the dressing would be prepared, and subjected to a whole range of tests e.g. bacteriological, microbiological, toxicity tests, and determinations made of its storage capability. The advantage of using a mixed acellular matrix is that unlike bioengineered substitutes incorporating living cells, the shelf life should be longer.

1 The publication of my UK patent entitled ‘A surgical-medical dressing for the treatment of body burns and for wound healing’ on Feb. 13^{th}, 2002 will arouse academic and commercial interest in HUVECs CM as well as my submitted 2001 paper.
If this dressing is to be used to treat a bums patient, it is envisaged that the whole dressing, will be enclosed in a gel-like substance, to facilitate easy handling by the attending physician. This gel-like substance will dissolve soon after being applied, i.e. contact with the patient's skin.
If this dressing is to be used in the treatment of a wound, it will be placed in the cavity of the wound and not removed. It is envisaged that several ‘strips’ of this dressing would be used depending on the size and depth of the wound.
It is envisaged that allogenic neonatal or infant keratinocytes (sparsely seeded) will be grown on the upper/outer layer of the dressing, if required for an ‘occlusive dressing’ use instead of an allograft/autograft.

2 Human vein endothelial cells (HUVECs) conditioned medium (CM) - - - HUVECs CM.
The first scientific report of the tissue culture of HUVECs was made by Jaffe et al. in 1973. This report described the perfusion of an umbilical cord with an enzyme, collagenase and the growth to confluence of HUVECs. Basic fibroblast growth factor (b-FGF) or commercially available endothelial cell growth supplements (ECGS) are added to the nutrient solution, (called the medium) to ensure that confluency is attained. There are scientific reports by Gospodarowicz et al. 1980a,b proving that assertion.
Little attention has been paid to the conditioned medium (CM) which is obtained after the nutrient medium is left in contact with the proliferating cells for periods of time which may vary from 2-3 days or longer. When the medium is changed, the conditioned medium is always discarded and an aliquot of fresh medium added.
In 1992, I published the first paper that showed a linkage between the extracellular secretions of HUVECs and human aortic endothelial cells. HUVECs were grown to post confluency (2 days after normal confluence) and the cell layer was detached with 5 mM EDTA (ethylene diamine tetra-acetic acid) leaving behind the sub-endothelial matrix (the so-called extracellular matrix (ECM)) intact. (The detached cells were reseeded onto a fresh substrate and re-grown time and again.)
Human aortic endothelial cells were obtained by mechanical scraping the intimal lining of a segment of human aorta, and these endothelial cells were grown to confluence on the HUVECs ECM, obtained as described above. A postulate was proposed that ECMs had a common phenotype.
In 2002, in a submitted paper to ‘The International Journal of Experimental Pathology’, I illustrated the fact that HUVECs CM may be used as a culture medium for human dermal microvascular endothelial cells (HDMECs), dermal fibroblasts, and epidermal keratinocytes, because of related extracellular secretions (see Table I above). The CM may be pre-screened by bacteriological and other tests before use as required.
DISPASE, a neutral protease is used to detach epithelial sheets (epidermal keratinocytes are grown in sheets in a tissue culture laboratory) which are used clinically as cultured epithelial autografts (CEAs) in the management of body bums.
Because CEAs are manufactured using Dispase, the ‘take’ of the graft is impaired (Rennekampff et al. 1996). The scientific paper describing Dispase was published in 1992. The title of the paper was: Dispase, a neutral protease from Bacillus polymxa, is a powerful fibronectinase and Type IV collagenase. HUVECs secrete into their conditioned medium (CM), Type IV procollagen, fibronectin and thrombospondin which makes the HUVECs CM a natural neutralizing agent for Dispase.
Cascade Biologics, Inc. (Portland, Oreg.), to name just one company in this line of business, sell all types of tissue culture media, cell types, ECGS etc. but not HUVECs CM. Interestingly, they sell pooled HUVECs, from more than one umbilical cord. The resultant ECM will not be pure as an original from one umbilical cord.
Scientific references:
Jaffe E. A., Nachman R. L., Becker C. G. & Minick C. (1973) Culture of human endothelial cells derived from umbilical veins. Identification by morphologic and immunologic criteria. J. Clin. Invest. 52, 2745-2756.
Gospodarowicz D., Delgado D. & Vlodavsky I. (1980a) Permissive effect of the extracellular matrix on cell proliferation in vitro. Proc. Natl. Acad. Sci. USA 77, 4094-4098.
Gospodarowicz D. & III C. (1980b) Extracellular matrix and control of proliferation of vascular endothelial cells. J. Clin. Invest. 65(6), 351-64.
Solomon D. E. (1992) The seeding of human aortic endothelial cells on the extra-cellular matrix of human umbilical vein endothelial cells. Int. J. Exp. Path. 73, 491-501.
Stenn K. S., Link R., Moellmann G., Madri J. & Kuklinska E. (1989) Dispase, a neutral protease from Bacillus polymxa, is a powerful fibronectinase and type IV collagenase. J. Invest. Dermatol. 93(2), 287-290.
Rennekampff H. O., Kiessig V. & Hansbrough J. F. (1996) Research Review-Current concepts in the development of cultured skin replacements. J. Surg. Res. 62, 288-295.
Solomon D. E. (2002) Submitted December 2001 - - - Int. J. Exp. Pathol. The inter-relationships between human endothelial cells, dermal fibroblasts and epidermal keratinocytes extracellular secretions, and their connection to wound healing.

3 By-product
A can spray of cornstarch and oil of cloves mixture.
Presently, a product called ‘Water.Jel’ (Carlstadt, N.J.) is used by City of Miami paramedics in onsite, first aid treatment of burn victims. My suggestion was initially an antibiotic-100% cornstarch spray in a durable container like a can spray. Enquiries have revealed that ‘Water.Jel’, a soothing wrap, suffers from fungi infection, and puncture of its packaging. Instead of using an antibiotic, and circumventing FDA approval, a mixture of oil of cloves and 100% cornstarch, packaged in a spray can is contemplated as a by-product.
Why cornstarch? It is a white granular carbohydrate occurring in the endosperm of corn kernel. It enjoys wide use as a major constituent of talcum body powder, in domestic cooking as a thickener, (a binding agent), for soups and stews and in the laundry product, spray starch. Cornstarch is used to soothe sunburn, for the relief of diaper rash, prickly heat, and itchy, irritated skin.
On arrival on hospital property, this white coating could be easily removed by salving the affected area. Alternatively, if maggots are to be used, they can be applied without salving. Anything that can stem tissue bleeding, loss of oozing wound fluid, and provide some small measure of pain relief, at the site of the incident, will be an improvement on ‘Water.Jel’.
This proposed spray should compromise the blood coagulation cascade, and the release of cellular growth factors e.g. PDGF and alpha-thrombin, which cause recruitment to the wound bed and both the proliferation and migration of fibroblasts.
It will also approximate an occlusive dressing, because there will be a vapor barrier. Minutes of the so-called ‘golden hour’ might be used beneficially, or even saved.
